(19) United States
(12) Patent Application Publication
(10) Pub. No.: US 2008/0253972 A1
(43) Pub. Date: Oct. 16, 2008

(54) Aqueous/alcoholic deodorant composition comprising a water-soluble zinc salt and salicylic acid

(76) Inventor: Estelle Prud'Homme, Paris (FR)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 11/902,086

(22) Filed: Sep. 19, 2007

Related U.S. Application Data
(60) Provisional application No. 60/847,390, filed on Sep. 27, 2006.

(30) Foreign Application Priority Data
Sep. 19, 2006 (FR) 0653810

(51) Int. Cl. A61K 8/27; A61K 33/30; A61Q 15/00

(52) U.S. Cl. 424/47; 424/642; 222/630

(57) ABSTRACT

The present disclosure relates to a deodorant composition comprising, in a cosmetically acceptable medium: a) at least one water-soluble zinc salt; b) salicylic acid; c) at least one C₁-C₅ monoalcohol; and d) at least 2% by weight of water, relative to the total weight of the composition. Another aspect of the present disclosure relates to the use of salicylic acid in a deodorant composition as stabilizing agent. The present disclosure also relates to a method of deodorizing, e.g., a method for treating human axillary odors, comprising applying an effective amount of said deodorant composition to a human axillary surface.

This application claims benefit of U.S. Provisional Application No. 60/847,390, filed Sep. 27, 2006, the contents of which are incorporated herein by reference. This application also claims benefit of priority under 35 U.S.C. § 119 to French Patent Application No. FR 0653810, filed Sep. 19, 2006, the contents of which are also incorporated herein by reference.

The present disclosure relates to a deodorant composition comprising, in a cosmetically acceptable medium,

a) at least one water-soluble zinc salt,

b) salicylic acid,

c) at least one C₁-C₅ monoalcohol,

d) at least 2% by weight of water, relative to the total weight of the composition.

It also relates to a cosmetic method for treating human axillary odors, comprising applying, to the axillary surface, an effective amount of the deodorant composition.

In the cosmetics field, it is known to use, in topical application, deodorant products comprising active substances of antiperspirant type or of bactericide type to reduce, and possibly eliminate, unpleasant axillary odors.

Bactericidal substances generally destroy resident bacteria flora. The most widely used among these substances are triclosan (2,4,4′-trichloro-2′-hydroxydiphenyl ether) and farnesol. Both of these substances, however, exhibit the disadvantage of significantly modifying the ecology of the cutaneous flora. Additional known bactericidal substances include those that reduce the growth of bacteria. Examples of these substances include transition metal chelating agents, such as EDTA or DPTA. These materials operate by depriving the surroundings of metals necessary for the growth of the bacteria. However, these active principles are potentially ecotoxic and may impose environmental problems.

In contrast to bactericidal compositions, antiperspirant substances operate by limiting the flow of sweat. These substances are generally composed of aluminum salts, which reduce the flow sweat by modifying the cutaneous physiology. However, these substances may irritate the skin, which is not satisfactory.

From this viewpoint, certain water-soluble zinc salts are already known as deodorant active principles in deodorant products, for instance in the form of water-in-silicone emulsions, such as, e.g., zinc pyrrolidonecarboxylate (more commonly referred to as zinc pidolate), zinc sulphate, zinc chlorate, zinc lactate, zinc gluconate and zinc phenolsulphonate. Such deodorant formulations have been described, for example, in International Patent Application No. WO 93/01793, and European Patent Application Nos. EP 468 564, EP 024 176, EP 768 080 and EP 1 486 199.

These water-soluble zinc salts have a very restricted bactericidal spectrum and, for example, a deodorant effectiveness unrelated to a bactericidal activity with regard to the microorganisms responsible for unpleasant odors. Further, when these zinc salts are formulated in an aqueous/alcoholic medium and applied to the skin, the solvents contained in the emulsion evaporate, thus leaving on the skin only compounds that do not have a bactericidal selectivity and do not risk inducing resistance in the bacteria. However, these aqueous/alcoholic formulations based on these water-soluble zinc salts have a tendency to destabilize and to form insoluble basic compounds, which precipitate and give an unattractive appearance.

International Patent Application No. WO 00/038644 also describes aqueous deodorant products which may comprise an alcohol solvent comprising a combination of zinc salt and a bicarbonate or carbonate salt stabilized by the presence of anions resulting from di-, tri- or polyacids or from di-, tri- or polyphosphates. However, these polyacid or polyphosphate anions may form complexes with the zinc ion, thereby greatly reducing its deodorant effectiveness.

Thus, it would be desirable to find novel aqueous/alcoholic formulations based on a water-soluble zinc salt which are effective with regard to deodorant activity, yet are without one or more of the disadvantages of the formulations of the prior art.

The present inventors have discovered, surprisingly, that, by adding salicylic acid to an aqueous/alcoholic formulation based on a water-soluble zinc salt, a composition is obtained which is stable, without formation of precipitate, and which has a good deodorant effectiveness unrelated to a bactericidal activity.

Therefore, one non-limiting aspect of the present disclosure is a deodorant composition comprising, in a cosmetically acceptable medium:

a) at least one water-soluble zinc salt;

b) salicylic acid;

c) at least one C₁-C₅ monoalcohol; and

d) at least 2% by weight of water, relative to the total weight of the composition.

As used herein, the term “deodorant composition,” means any composition capable of producing a reduction in the unpleasant odors related to the decomposition of sweat.

Another non-limiting aspect of the present disclosure is the use of salicylic acid in a deodorant composition comprising, in a cosmetically acceptable medium:

(a) at least one water-soluble zinc salt,

(b) at least 2% by weight of water, relative to the total weight of the composition, and

(c) at least one C₁-C₅ monoalcohol,

as stabilizing agent for the composition.

Finally, another non-limiting aspect of the present disclosure is a deodorizing method employing the presently disclosed deodorant composition, e.g., a method for treating human axillary odors comprising applying, to the axillary surface, an effective amount of the deodorant composition.

As used herein, the term “water-soluble zinc salt,” means any salt which, after having been completely dissolved with stirring at 1% in an aqueous solution at a temperature of 25° C., results in a solution comprising less than 0.05% by weight of an insoluble salt.

As examples of such water-soluble zinc salts, non-limiting mention may be made of zinc pyrrolidonecarboxylate (more commonly referred to as zinc pidolate), zinc sulphate, zinc chloride, zinc lactate, zinc gluconate, zinc phenolsulphonate, zinc salicylate and its derivatives, and mixtures thereof.

Non-limiting mention may also be made of zinc salicylate and its derivatives, which, according to the present disclosure, correspond to the following structure:

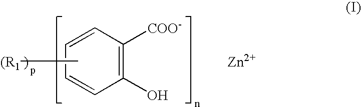

in which n is 2, p is an integer ranging from 0 to 3, and R₁ is chosen from linear or branched C₁-C₁₈ alkyl radicals (for example methyl, ethyl, n-propyl, isopropyl or n-butyl); linear or branched C₁-C₁₈ hydroxyalkyl radicals; halogen atoms (for example, iodine, bromine, chlorine); C₂-C₁₈ acyl radicals (for example, acetyl); and COR₂, OCOR₂ and CONHR₂ radicals, wherein R₂ is a hydrogen atom or a linear or branched C₁-C₁₈ alkyl radical.

For example, in at least one embodiment, the water-soluble zinc salt is zinc salicylate (p=0), for example, in the hydrate form, such as zinc salicylate trihydrate, such as the commercial product sold under “Zinc Salicylate Trihydrate” by Bernardy Chimie.

In the deodorant compositions according to the present disclosure, the water-soluble zinc salt may be present in an amount ranging from 0.1 to 5% by weight, for example, from 0.1 to 2% by weight, relative to the total weight of the composition.

Salicylic acid may be present in the deodorant composition in an amount ranging from 0.01 to 0.5% by weight, for example, from 0.05 to 0.2% by weight, relative to the total weight of the composition.

The deodorant compositions of the present disclosure may be formulated by conventional methods such that they are suitable for the applications for which they are intended.

The C₁-C₅ monoalcohol or monoalcohols present in the compositions of the present disclosure may be chosen from, for example, methanol, ethanol, isopropanol and mixtures thereof. In at least one embodiment, the C₁-C₅ monoalcohol is ethanol. The C₁-C₅ monoalcohol or monoalcohols may be present in the composition in an amount ranging from greater than 10% by weight, for example, from 15 to 96% by weight, relative to the total weight of the composition.

Water may be present in the composition in an amount ranging from 2 to 85% by weight, for example, from 2 to 30% by weight, relative to the total weight of the composition.

The deodorant compositions according to the present disclosure intended for cosmetic use can be provided in the form of: lotions dispensed in vaporizers or aerosol pumps, creams dispensed in a tube or in a twist stick, or gels distributed in a roll-on device or in a twist stick. The deodorant compositions of the present disclosure may also comprise ingredients generally used in products of this type and which are known in the art, with the proviso that these additional ingredients do not interfere with the water-soluble zinc salt and the salicylic acid described above.

The deodorant compositions according to the present disclosure intended for cosmetic use can be provided in the form of an emulsion, such as, for example, a water-in-oil emulsion, an oil-in-water emulsion or a multiple emulsion (oil-in-water-in-oil or water-in-oil-in-water triple emulsion). Such emulsions are described, for example, by C. FOX in “Cosmetics and Toiletries”, November 1986, Vol. 101, pages 101-112.

The compositions according to the present disclosure may also comprise a water-immiscible organic liquid phase. This water-immiscible organic liquid phase comprises at least one hydrophobic compound that renders the phase immiscible in water. In the absence of structuring agents, the water-immiscible organic phase is liquid at ambient temperature (e.g., 20-25° C.). In at least one embodiment, the water-immiscible organic liquid phase disclosed herein comprises an oil or a mixture of oils, and further comprises at least 80% of compounds having a vapor pressure of 4 kPa (30 mmHg) or less at 25° C.

In a further non-limiting embodiment, the water-immiscible organic liquid phase comprises at least one volatile or nonvolatile silicone or hydrocarbon emollient oil. These emollient oils are described U.S. Pat. Nos. 4,822,596 and 4 904 463.

As used herein, the term “volatile silicone” refers to silicone compounds that are volatile at ambient temperature. As non-limiting examples of volatile silicones, mention may be made of cyclic and linear volatile silicones of the dimethylsiloxane type, the chains of which comprise from 3 to 9 silicone residues. In at least one embodiment, the volatile silicone is chosen from D4, D5 and D6 cyclomethicones.

As used herein, the term “non-volatile silicone” refers to compounds having a low vapor pressure at ambient temperature. Non-limiting examples of non-volatile silicones include polyalkylsiloxanes, for example linear polyalkylsiloxanes, such as, for example, the linear polydimethylsiloxanes or dimethicones sold by Dow Corning under the name of “Dow Corning 245 Fluid”; polyalkylarylsiloxanes, such as, for example, the polymethylphenylsiloxanes sold by Dow Corning under the name of “Dow Corning 556 Fluid”; or polyether and siloxane copolymers, such as, for example, dimethicone copolyols.

Mention may be made, among the non-volatile emollient oils which can be used in the present disclosure, of, for example: hydrocarbon derivatives, mineral oils, fatty alcohols, esters of C₃-C₁₈ alcohols with C₃-C₁₈ acids, esters of benzoic acid with C₁₂-C₁₈ alcohols and mixtures thereof, and C₂-C₆ polyols, for example, C₂-C₆ polyols chosen from glycerol, propylene glycol sorbitol, or polyalkylene glycol polymers.

The emollient oils may be present in the composition in an amount ranging from 1 to 50% by weight, for example, from 5 to 40% by weight, relative to the total weight of the composition.

The deodorant cosmetic composition according to the present disclosure may also comprise at least one additional deodorant active principle, such as, but not limited to, bacteriostatic agents or bactericidal agents, such as 2,4,4′-trichloro-2′-hydroxydiphenyl ether (triclosan), 2,4-dichloro-2′-hydroxydiphenyl ether, 3′,4′,5′-trichlorosalicylanilide, 1-(3′,4′-dichlorophenyl)-3-(4′-chlorophenyl)urea (triclocarban) or 3,7,11-trimethyldodeca-2,5,10-trienol (farnesol); quaternary ammonium salts, such as cetyltrimethylammonium salts or cetylpyridinium salts; chlorhexidine and its salts; diglycerol monocaprate, diglycerol monolaurate or glycerol monolaurate; or polyhexamethylene biguanide salts.

The deodorant cosmetic composition according to the present disclosure may also comprise at least one antiperspirant aluminum salt.

As used herein, the term “antiperspirant aluminum salt” means any aluminum salt or any aluminum complex having the effect of reducing or limiting the flow of sweat.

In at least one embodiment, the aluminum salts that may be used in accordance with the present disclosure are chosen from aluminum halohydrates; aluminum zirconium halohydrates, and complexes of zirconium chlorohydrate and of aluminum chlorohydrate, with or without an amino acid, e.g., those described in U.S. Pat. No. 3,792,068.

Mention may further be made, among the aluminum salts disclosed herein, of aluminum chlorohydrate in the activated or nonactivated form, aluminum chlorohydrex, the aluminum chlorohydrex polyethylene glycol complex, the aluminum chlorohydrex propylene glycol complex, aluminum dichlorohydrate, the aluminum dichlorohydrex polyethylene glycol complex, the aluminum dichlorohydrex propylene glycol complex, aluminum sesquichlorohydrate, the aluminum sesquichlorohydrex polyethylene glycol complex, the aluminum sesquichlorohydrex propylene glycol complex, or aluminum sulphate buffered by sodium aluminum lactate.

With respect to aluminum zirconium salts, non-limiting mention may be made of aluminum zirconium octachlorohydrate, aluminum zirconium pentachlorohydrate, aluminum zirconium tetrachlorohydrate and aluminum zirconium trichlorohydrate.

The complexes of zirconium chlorohydrate and of aluminum chlorohydrate with an amino acid are generally known under the name ZAG (where the amino acid is glycine). Non-limiting examples of such compounds include the aluminum zirconium octachlorohydrex glycine, aluminum zirconium pentachlorohydrex glycine, aluminum zirconium tetrachlorohydrex glycine and aluminum zirconium trichlorohydrex glycine complexes.

In at least one embodiment of the present disclosure, aluminum chlorohydrate is used in the deodorant composition in the activated or nonactivated form.

The antiperspirant aluminum salts may be present in the composition in an amount ranging from 0.5 to 25% by weight, relative to the total weight of the composition.

The composition of the present disclosure may also comprise, to improve the homogeneity of the product, at least one suspending agent. In a non-limiting embodiment of the present disclosure, the at least one suspending agent is chosen from hydrophobic modified montmorillonite clays, such as hydrophobic modified bentonites or hectorites. Mention may be made, for example, of the product Stearalkonium Bentonite (CTFA name) (reaction product of bentonite and of the quaternary ammonium stearalkonium chloride), such as the commercial product sold under the name Tixogel MP 250 by Sud Chemie Rheologicals, United Catalysts Inc., and the product Disteardimonium Hectorite (CTFA name) (reaction product of hectorite and of distearyidimonium chloride), sold under the name of Bentone 38 or Bentone Gel by Elementis Specialities.

The suspending agents may be present in the composition in an amount ranging from 0.1 to 5% by weight, for example, from 0.2 to 2% by weight, relative to the total weight of the composition.

The compositions according to the present disclosure may further comprise at least one filler. Mention may be made, among the fillers which can be used according to the present disclosure, of organic powders. As used herein, the term, “organic powder” means any solid which is insoluble in the cosmetically acceptable medium at ambient temperature (25° C.).

Mention may be made, as organic powders which can be used in the composition of the disclosure, for example, of
polyamide particles, e.g., those sold under the name Orgasol by Atochem; polyethylene powders;
microspheres based on acrylic copolymers, e.g., those made of ethylene glycol dimethacrylate/lauryl methacrylate copolymer and sold by Dow Corning under the name Polytrap;
poly(methyl methacrylate) microspheres sold under the name Microsphere M-100 by Matsumoto or under the name Covabead LH85 by Wacker;
powders formed of ethylene/acrylate copolymer, e.g., those sold under the name Flobeads by Sumitomo Seika Chemicals;
expanded powders such as hollow microspheres, e.g., the microspheres formed from a terpolymer of vinylidene chloride, acrylonitrile and methacrylate and sold under the name Expancel by Kemanord Plast under the references 551 DE 12 (particle size of approximately 12 μm and density 40 kg/m³), 551 DE 20 (particle size of approximately 30 μm and density 65 kg/m³), or 551 DE 50 (particle size of approximately 40 μm), or the microspheres sold under the name Micropearl F 80 ED by Matsumoto;
powders formed from natural organic materials, such as powders formed from starch, e.g., powders formed from crosslinked or noncrosslinked maize, wheat or rice starches, such as the powders formed from starch crosslinked by octenylsuccinic anhydride and sold under the name Dry-Flo by National Starch;
silicone resin microbeads, such as those sold under the name Tospearl by Toshiba Silicone, e.g., Tospearl 240;
powders formed from amino acids, such as the lauroyllysine powder sold under the name Amihope LL-11 by Ajinomoto;
wax microdispersion particles, including those having a mean particle size of less than 1 μm, for example from 0.02 μm to 1 μm, and which comprise a wax or of a mixture of waxes, e.g., the products sold under the name Aquacer by Byk Cera, for example: Aquacer 520 (mixture of synthetic and natural waxes), Aquacer 514 or 513 (polyethylene wax) or Aquacer 511 (polymer wax), or the product sold under the name Jonwax 120 by Johnson Polymer (mixture of polyethylene and paraffin waxes) and under the name Ceraflour 961 by Byk Cera (micronized modified polyethylene wax); and
mixtures thereof.

The cosmetic composition according to the present disclosure may also comprise cosmetic adjuvants chosen from waxes, softeners, antioxidants, opacifiers, stabilizers, moisturizing agents, vitamins, fragrances, bactericides, preservatives, polymers, thickening agents, propellants or any other ingredient commonly used in cosmetics in this type of application.

Of course, a person skilled in the art will take care to choose the aforementioned optional additional compounds so that the advantageous properties intrinsically attached to the cosmetic composition in accordance with the present disclosure are not, or not substantially, detrimentally affected by the envisaged addition or additions.

As non-limiting examples of waxes that may be used in accordance with the present disclosure, mention may be made of animal, fossil, vegetable, mineral or synthetic waxes. Of these, for example, further mention may be made of beeswaxes, carnauba, candelilla, sugarcane or japan waxes, ozokerites, montan wax, microcrystalline waxes, paraffin waxes or silicone waxes and resins.

As non-limiting examples of thickeners that may be used in accordance with the present disclosure, mention may be made of modified or unmodified guar gums and celluloses, such as hydroxypropylated guar gum, cetylhydroxyethylcellulose or silicas, such as, Bentone Gel MIO, sold by NL Industries, or Veegum Ultra, sold by Polyplastic. In at least one embodiment, the thickeners are nonionic.

The amounts of these various constituents which can be present in the cosmetic composition according to the present disclosure are those amounts conventionally used in deodorant compositions.

The compositions according to the present disclosure can also comprise at least one structuring or gelling agent for the water-immiscible organic liquid phase of the composition, such as waxes and/or linear solid fatty alcohols; fatty acids or their salts (e.g., stearic acid, sodium stearate, 12-hydroxystearic acid); dibenzylidene alditols (e.g., DBS); lanosterol; N-acylamino acid derivatives; derivatives of di- or tricarboxylic acids, such as alkyl-N,N′-dialkylsuccinamides (e.g., dodecyl-N,N′-dibutylsuccinamide); and organopolysiloxane elastomers, for example, those described in International Patent Application WO 97/44010.

The composition according to the present disclosure can also be pressurized and be packaged in an aerosol device.

The present disclosure therefore also relates to an aerosol device comprising:

(A) a container comprising a deodorant composition as defined above, and

(B) at least one propellant and a means for dispensing the said aerosol composition.

The propellants useful herein are those generally used in products of this type and which are well known to a person skilled in the art, such as, for example, dimethyl ether (DME), and volatile hydrocarbons, for example, n-butane, propane or isobutane, and mixtures thereof, and optionally comprising at least one chlorinated and/or fluorinated hydrocarbon. Non-limiting mention may be made, among the latter, of the compounds sold by Dupont de Nemours under the names Freon® and Dymel®, including, for example, monofluorotrichloromethane, difluorodichloromethane, tetrafluorodichloroethane and 1,1-difluoroethane, sold, e.g., under the trade name Dymel 152 A by DuPont. Further non-limiting examples of propellants include carbon dioxide gas, nitrous oxide, nitrogen and compressed air.

The deodorant active principle or principles and the propellant or propellants may be present in the same or in different compartments of the aerosol container.

According to the present disclosure, the propellant may be present in an amount ranging from 5 to 95% by weight pressurized, for example, from 50 to 85% by weight pressurized, relative to the total weight of the pressurised composition.

The aerosol device may also comprise a dispensing means, which itself may comprise a dispensing valve controlled by a dispensing head, the dispensing head comprising a nozzle via which the aerosol composition is vaporized. The aerosol device may also comprise a container for containing the pressurized composition, said container being opaque or transparent. The container can be made, for example, of glass, of polymer or of metal, and may optionally be covered with a layer of protective lacquer.

The present disclosure further relates to a cosmetic method for treating human axillary odors, comprising applying, to the axillary surface, an effective amount of a composition as defined above.

Other than in the examples, or where otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present disclosure. At the very least, and not as an attempt to limit the application of the doctrine of equivalents to the scope of the claims, each numerical parameter should be construed in light of the number of significant digits and ordinary rounding approaches.

Notwithstanding that the numerical ranges and parameters setting forth the broad scope of the present disclosure are approximations, unless otherwise indicated the numerical values set forth in the specific examples are reported as precisely as possible. Any numerical value, however, inherently contain certain errors necessarily resulting from the standard deviation found in their respective testing measurements.

By way of non-limiting illustration, concrete examples of certain embodiments of the present disclosure are given below. The amounts of the ingredients are expressed as percentages by weight, relative to the total weight of the composition.

### EXAMPLES

### Examples 1 and 2 of Aqueous/Alcoholic Zinc Pidolate Solutions and Stability on Storage after Two Months in an Oven at 45° C.

| | Example 1 | Example 2 |
|---|---|---|
| Ingredients | (comparative) | (inventive) |
| 96% ethanol by volume | 20 | 20 |
| (4% of water) | | |
| Zinc pidolate | 0.5 | 0.5 |
| Salicylic acid | — | 0.055 |
| Water | q.s. for 100 | q.s. for 100 |
| Appearance after storing | Clear liquid with | Clear liquid without |
| at 45° C. for 2 months | precipitates | precipitates |

### Examples 3 to 5 of Alcoholic-Based Zinc Salicylate Aerosols and Stability on Storage after Two Months in an Oven at 45° C.

| | Example 3 | | | Example 6 |
|---|---|---|---|---|
| | (compar- | Example 4 | Example 5 | (compar- |
| Ingredients | ative) | (inventive) | (inventive) | ative) |
| 96% ethanol by | 42.9 | 42.9 | 44.4 | 44.5 |
| volume (4% of | | | | |
| water) | | | | |
| Zinc salicylate | 0.60 | 0.47 | 0.50 | 0.47 |
| Salicylic acid | — | 0.13 | 0.10 | — |
| Fragrance | 1.5 | 1.5 | — | — |
| Isobutane | 55 | 55 | 55 | 55 |
| Appearance | Clear liquid | Clear liquid | Clear liquid | Clear liquid |
| after storing at | with | without | without | with |
| 45° C. for 2 | precipi- | precipitate | precipitate | precipi- |
| months | tates | | | tates |

Test of In Vivo Effectiveness of the Alcoholic Aerosol of Example 7 in Comparison with a Triclosan-Based Alcoholic Aerosol

The following alcoholic aerosol formulations were tested:

| | Example 7 | Example 8 |
|---|---|---|
| Ingredients | (inventive) | (comparative) |
| Denatured 96% | 44.4% | 44.8% |
| ethyl alcohol | | |
| Zinc salicylate | 0.47% | — |
| Salicylic acid | 0.12% | — |
| Triclosan | — | 0.15 |
| Isobutane | 55.0 | 55.0 |

A sensory test was carried out on a panel of 20 volunteers. After wiping the armpit of each volunteer, a composition was applied once per armpit in an amount of 1.2±0.05 g vaporized at 15 cm from the armpit. A panel of five experts then evaluated:

(1) the intensity of the odor of perspiration, 8 hours after application, and assigned a grade from 1 to 9 (1: odor of perspiration imperceptible and 9: odor extremely strong); and

(2) the intensity of the annoyance or the hedonic value, 8 and 24 hours after application, and assigned a grade from 1 to 9 (1: odor extremely unpleasant and 9: extremely pleasant). The results obtained are summarized in the following table:

Results 8 Hours after Application

| | Intensity of the odor of | | | |
|---|---|---|---|---|
| | perspiration | | Hedonic value of the odor | |
| | Armpit treated with | Armpit treated | Armpit treated | Armpit treated |
| | Example 7 | with Example 8 | with Example 7 | with Example 8 |
| Mean | 3.9 | 3.7 | 3.7 | 3.7 |
| Standard | 1.8 | 1.3 | 1.1 | 1.0 |
| deviation | | | | |
| % of | +5% | +5% | 0% | 0% |
| variation | | | | |

Results 24 Hours after Application

| | Intensity of the odor of | | | |
|---|---|---|---|---|
| | perspiration | | Hedonic value of the odor | |
| | Armpit | Armpit | Armpit | Armpit |
| | treated with | treated with | treated with | treated with |
| | Example 7 | Example 8 | Example 7 | Example 8 |
| Mean | 4.1 | 4.3 | 3.6 | 3.5 |
| Standard | 1.8 | 1.6 | 1.1 | 1.1 |
| deviation | | | | |
| % of | −5% | −5% | +4% | +4% |
| variation | | | | |

The composition of Example 7, which is in accordance with the present disclosure, exhibited a deodorant effectiveness equivalent to that of comparative Example 8.

Demonstration of the Bactericidal Activity of the Zinc Salicylate/Salicylic Acid Combination with Regard to the Microorganisms Involved in Axillary Odors

The test described here makes possible the quantitative determination of the bactericidal activity of an active principle (i.e.: zinc salicylate/salicylic acid or triclosan) conveyed in an appropriate carrier which is neutral with regard to the bacteria tested. The bacteria studied were microorganisms under optimum growth conditions, namely microorganisms of the Corynebacterium xerosis type (Institut Pasteur Collection No. 5216) cultured on a gradient of tryptocasein soybean agar.

The day before the test, 32 g of tryptocasein soybean broth was placed in a sample tube and incubated at 35° C. The day of the test, 4 g of the test composition was added and the mixture was homogenized using a Vortex render.

A growth control without product was also prepared under the same conditions in order to confirm that the microorganisms are under favorable growth conditions throughout the duration of the test.

For the preparation of the inoculum, five days before the start of the test, the two bacterial strains were subcultured on suitable medium. They were incubated at 35° C. for 5 days. On the day of the test, the gradient was washed with approximately 9 ml of diluent. The suspension obtained had a titre of 108 microorganisms/ml (counting is carried out). 4 ml of inoculum was introduced into the sample tube, which corresponded to a level of 107 bacteria per gram of preparation. The sample tube was then placed in an incubator/sitter (35° C., 200 rpm).

After a contact time of 2, 4, 6 and 24 hours, the contents of each sample tube were homogenized using a Vortex render. Ten fold dilutions were carried out. The contents were placed on the surface of agar Petri dishes (Eugon LT 100 medium). The Petri dishes were then incubated in an oven at 35° C. for 6 to 7 days.

The colonies on the dishes comprising more than 20 and less than 200 colonies were counted.

With respect to the evaluation of the active principle, the results were expressed, for each microorganism tested, as the Log number of reduction after 24 hours with respect to the control/placebo, as follows:

| | |
|---|---|
| Identical results: | none, |
| 1 Log of reduction: | low, |
| 2 Log of reduction: | moderate, |
| 3 Log of reduction: | good, |
| ≧4 Log of reduction: | excellent. |

The following two carrier gels A and B were prepared (amounts expressed as percentages by weight):

| | Carrier A | Carrier B |
|---|---|---|
| Ingredients | (invention) | (prior art) |
| Triclosan | | 0.1% |
| Zinc salicylate | 0.47% | |
| Salicylic acid | 0.12% | |
| Polyethylene glycol, 8 EO | 6% | 6% |
| Crosslinked acrylic | 0.9% | 0.9% |
| acid/C₁₀-C₃₀ alkyl acrylate | | |
| copolymer | | |
| Sodium hydroxide, pure | q.s. (pH = 7) | q.s. (pH = 7) |
| Deionized water, | q.s. for 100% | q.s. for 100% |
| microbiologically clean | | |

The bactericidal activity of each of formulations 9 and 10 (see below) with regard to the Corynebacterium xerosis strain was measured and compared to a formulation devoid of active principle.

The results obtained are summarized in the following table:

| | Effectiveness at 24 hours with |
|---|---|
| | respect to the gel devoid of active |
| Active principle tested | principle |
| Strains | Corynebacterium xerosis |
| Zinc salicylate and salicylic acid | Low |
| Triclosan | Excellent |

The zinc salicylate/salicylic acid combination had a low antibacterial activity with regard to the Corynebacterium Xerosis strains. It thus had a substantially narrower spectrum of bactericide activity than that of triclosan and is more respectful of the cutaneous flora.

### Examples 9 and 10 of Aqueous/Alcoholic Solutions Based on Zinc Salicylate and on Carboxylic Acid

| | Example 9 | Example 10 |
|---|---|---|
| Ingredients | (comparative) | (inventive) |
| Zinc salicylate | 0.91 | 0.91 |
| Citric acid | 0.05 | — |
| Salicylic acid | — | 0.05 |
| 96% ethanol by volume | q.s. for 100 | q.s. for 100 |
| (4% of water) | | |
| Immediate appearance | Formation of white | Absence of white |
| after manufacture | precipitate | precipitate |

What is claimed is:

1. A deodorant composition comprising, in a cosmetically acceptable medium:
a) at least one water-soluble zinc salt,
b) salicylic acid,
c) at least one C₁-C₅ monoalcohol, and
d) at least 2% by weight of water, relative to the total weight of the composition.

2. The deodorant composition of claim 1, wherein said at least one water-soluble zinc salt is chosen from zinc pidolate, zinc sulphate, zinc chloride, zinc lactate, zinc gluconate, zinc phenolsulphonate, zinc salicylate and its derivatives, and mixtures thereof.

3. The deodorant composition of claim 2, wherein the zinc salicylate and its derivatives correspond to the following structure: 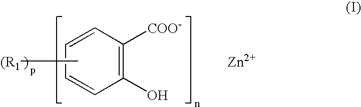
in which:
n is equal to 2;
p is an integer ranging from 0 to 3; and
R₁ is chosen from linear or branched C₁-C₁₈ alkyl radicals, linear or branched C₁-C₁₈ hydroxyalkyl radicals, halogen atoms, C₂-C₁₈ acyl radicals, and COR₂, OCOR₂ and CONHR₂ radicals, wherein R₂ is chosen from a hydrogen atom and a linear or branched C₁-C₁₈ alkyl radical.

4. The deodorant composition of claim 3, where the water-soluble zinc salt is zinc salicylate.

5. The deodorant composition of claim 1, wherein the at least one water-soluble zinc salt is present in an amount ranging from 0.1 to 5% by weight, relative to the total weight of the composition.

6. The deodorant composition of claim 1, wherein the salicylic acid is present in an amount ranging from 0.01 to 0.5% by weight, relative to the total weight of the composition.

7. The deodorant composition of claim 1, wherein the at least one C₁-C₅ monoalcohol is chosen from methanol, ethanol, isopropanol, and mixtures thereof.

8. The deodorant composition of claim 1, wherein the at least one C₁-C₅ alcohol is ethanol.

9. The deodorant composition of claim 1, wherein the at least one C₁-C₅ monoalcohol is present in the composition at a concentrations of greater than 10% by weight, relative to the total weight of the composition.

10. The deodorant composition of claim 1, where the water is present in the composition in an amount ranging from 2 to 85% by weight, relative to the total weight of the composition.

11. The deodorant composition of claims 1, wherein said composition is provided in the form of:
a lotion dispensed in a vaporizer or aerosol pump;
a cream dispensed in a tube or in a twist stick; or
a gel dispensed in a roll-on device or in a twist stick.

12. The deodorant composition according to claim 1, wherein said composition is provided in the form of an emulsion.

13. The deodorant composition of claim 1, further comprising a water-immiscible organic liquid phase.

14. The deodorant composition of claim 13, wherein the water-immiscible organic liquid phase comprises at least one oil chosen from volatile silicone oils, nonvolatile silicone oils, and hydrocarbon emollient oils.

15. The deodorant composition of claim 14, wherein said at least oil is chosen from emollient oils and is present in the composition in an amount ranging from 1 to 50% by weight relative to the total weight of the composition.

16. The deodorant composition of claim 1, further comprising at least one additional deodorant active principle.

17. The deodorant composition of claim 1, further comprising at least one antiperspirant aluminum salt.

18. The deodorant composition of claim 1, further comprising at least one suspending agent.

19. The deodorant composition of claim 1, further comprising at least one filler.

20. The deodorant composition of claim 1, further comprising at least one cosmetic adjuvant chosen from waxes, softeners, antioxidants, opacifiers, stabilizers, moisturizing agents, vitamins, fragrances, bactericides, preservatives, polymers, thickening agents and propellants.

21. The deodorant composition of claim 13, further comprising at least one other structuring or gelling agent for the water-immiscible organic liquid phase.

22. An aerosol device, comprising:
(A) a container comprising a deodorant composition, said deodorant composition comprising, in a cosmetically acceptable medium:
i) at least one water-soluble zinc salt,
ii) salicylic acid,
iii) at least one C₁-C₅ monoalcohol, and
iv) at least 2% by weight of water, relative to the total weight of the composition; and
(B) at least one propellant and a means for dispensing the said aerosol composition.

23. A method for stabilizing a deodorant composition, said method comprising including salicylic acid as a stabilizing agent in a deodorant composition comprising, in a cosmetically acceptable medium:
(a) at least one water-soluble zinc salt,
(b) at least 2% by weight of water, with respect to the total weight of the composition, and
(c) at least one C₁-C₅ monoalcohol.

24. A method for treating human axillary odors, comprising applying to a human axillary surface an effective amount of a deodorant composition comprising, in a cosmetically acceptable medium:
i) at least one water-soluble zinc salt,
ii) salicylic acid,
iii) at least one C₁-C₅ monoalcohol, and
iv) at least 2% by weight of water, relative to the total weight of the composition.
